(19) United States
(12) Patent Application Publication
(10) Pub. No.: US 2010/0190709 A1
(43) Pub. Date: Jul. 29, 2010

(54) Angiogenic composition

(75) Inventors: Olivier Soula, Meyzieu (FR); Gerard Soula, Meyzieu (FR); Remi Soula, Lyon (FR); Rosy Eloy, Ternay (FR)

Correspondence Address: OLIFF & BERRIDGE, PLC, P.O. BOX 320850, ALEXANDRIA, VA, 22320-4850, US

(73) Assignee: ADOCIA, LYON (FR)

(21) Appl. No.: 12/659,891

(22) Filed: Mar. 24, 2010

Related U.S. Application Data
(63) Continuation of application No. 12/078,443, filed on Mar. 31, 2008, now abandoned.
(60) Provisional application No. 60/907,368, filed on Mar. 29, 2007.

(30) Foreign Application Priority Data
Mar. 29, 2007 (FR) 07 02314

(51) Int. Cl. A61K 38/18; A61P 7/00

(52) U.S. Cl. 514/12

(57) ABSTRACT

The present invention relates to a amphiphilic polymer in the preparation of a therapeutic composition for promoting angiogenesis at its site of administration, comprising a complex between a polymer and a PDGF, wherein the polymer is amphiphilic.

In an embodiment, the PDGF is selected from the group of the PDGFs (platelet-derived growth factors) and the amphiphilic polymer is selected from the group:

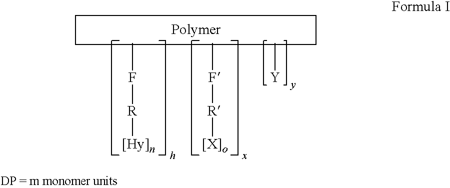

The invention relates also to the therapeutic composition is in the form of a gel, a cream, a solution, a spray, a paste or a patch or a dressing.

This is a Continuation of application Ser. No. 12/078,443 filed Mar. 31, 2008, which claims the benefit of French Application No. 07 02314 filed Mar. 29, 2007 and U.S. Provisional Application No. 60/907,368. The entire disclosure of the prior applications is hereby incorporated by reference herein in their entirety.

The present invention relates to a novel angiogenic treatment based on PDGF, platelet-derived growth factor.

The invention can be used in the treatment of problems of ischemia, especially peripheral ischemia, such as ischemia of a lower limb, eschars, venous ulcers, compression ulcers, myocardial ischemia, colitis, Raynaud's syndrome, osteonecrosis of the femoral head, certain ophthalmic problems of vascular origin, ischemia of the optic papilla, corneal ulcerations, and certain complications that arise in the case of diabetes, in particular ulcerations of the diabetic foot.

Angiogenesis represents a major therapeutic challenge. On the one hand it is sometimes vital to revascularize organs and tissues, and on the other hand there is no pharmacological means of creating new vessels. The only therapeutic agents available are vasodilatory agents, which temporarily increase the diameter of and/or the flow through existing vessels. Even today, the creation of a vessel de novo is a difficult objective which has not yet been achieved, and when the blood flow is reduced owing to lesions of the vascular wall (atheroma, atherosclerosis), vascular surgery allows a flow to be established beneath the lesion by means of a derivation or bypass, using vascular prostheses or grafts (made of synthetic or biological materials, respectively). Only vessels having a diameter equal to or greater than 4 mm are amenable to such replacements, despite the use of microsurgical techniques. The peripheral revascularization of tissues, which depend on capillaries several hundred microns in diameter, cannot therefore be envisaged by such surgical techniques, and only the stimulation of the growth of neovessels or angiogenesis can be envisaged.

Today, angiogenesis is very well described on the scientific plane, and the growth factors involved are well known, including inter alia, in order of importance, VEGF, TNFα, TGFα, thrombin, proliferin, PDGF, MMP-1, MMP-2, MMP-9, IL-1, IL-4, IL-6, IL-8 and IL-13. Many scientific, academic and industrial groups are working at using those proteins therapeutically. The value of two of those growth factors has been demonstrated clinically.

The most effective of those angiogenic growth factors is VEGF, vascular endothelial growth factor VEGF (Pandya, N. M. et al., Vascul. Pharmacol. 2006, 44 (5), 265-274). That growth factor has recently been tested on a diabetic mouse wound model by Genentech (Galiano, Robert D. et al., Am. J. Pathol. 2004, 164 (6), 1935-1947). That growth factor exhibits an effectiveness that is far superior to that of the control in this model, both in terms of formation of the granulation tissue, neovascularization, and scarring time. The results confirm the importance of neovascularization in the scarring process. VEGF is developed by Genentech for the treatment of diabetic foot ulcers. The results of clinical phase I/II have shown a level of scarring of 41% at the end of 6 weeks, compared with 26% for conventional treatments without growth factor. However, VEGF has not been approved to date and risks of uncontrolled vascularization are possible. Furthermore, it has been shown that VEGF is an angiogenesis initiator but is not sufficient for the formation of a mature vascular system (Yancopoulos, G. D. et al., Nature 2000, 407 (6801), 242-248). Angiogenesis with VEGF is therefore provisional.

PDGF is the only growth factor that is approved in the indication of scarring. It is produced by genetic recombination and is marketed by Johnson & Johnson under the name Regranex for the treatment of diabetic foot ulcers.

In the dossier submitted by Johnson & Johnson for the approval of Regranex (Tiwari, Jawahar, PLA 96-1408 REGRANEX (becaplermin) Gel (recombinant human platelet-derived growth factor) in the treatment of diabetic foot ulcers. 5 Sep. 1997, Food and Drug Administration), it is interesting to note that, during clinical trials, Johnson & Johnson described an angiogenic ability without being able to demonstrate a real dose-related effect beyond 0.01%, probably because of a lack of solubility.

PDGF-BB administered by gene therapy confirms the angiogenic potential of that growth factor.

The administration of PDGF by gene therapy in fact gives much better results on angiogenesis. Genes coding for PDGF-B are administered by an adenovector formulated in a collagen matrix. That gene therapy, Excellarate, is developed by Tissue Repair Company, recently acquired by Cardium Therapeutics. The method has the advantage of maintaining PDGF production at the site of the wound for a relatively long time. Application of that product to the wound of a diabetic mouse model showed that granulation, neovascularization and epithelization are strongly stimulated (Keswani, Sundeep G. et al., Wound Repair Regen. 2004, 12, 497-504).

Other Japanese researchers, Y. Yonemitsu's team, have shown that microangiopathy of the lower limb in diabetics is a disease that is caused by a disturbance of the PDGF-BB/protein kinase C pair and is not due to a lack of expression of other angiogenic factors, in particular VEGF, HGF, FGF-2, angiopoietin-1 and -2 (Tanii, Mitsugu et al., Circ. Res. 2006, 98, 55-62). In those works, the approach is again gene therapy.

However, gene therapy is more difficult to develop in the near future because of its potential risks in particular owing to the non-selective transfection of cells.

Another type of administration of PDGF has been published by Hsieh P. et al. They are formulations of PDGF-BB with a synthetic oligopeptide capable of forming nanofibers which are injected into the myocardium (Hsieh, P. C. et al., J. Clin. Invest. 2006, 116 (1), 237-248) (Hsieh, Patrick C. H. et al., Circulation 2006, 114, 637-644). Their technique permits the release of PDGF over 14 days. Regeneration of the myocardium has been obtained in a rat model bearing an infarct. According to the same authors, in that formulation, the nanofibers appear to have intrinsic angiogenic ability (Narmoneva, Daria A. et al., Biomaterials 2005, 26, 4837-4846).

Accordingly, it appears that PDGF, owing to its intrinsic angiogenic activity and its non-toxic nature, which is proven after years of use in patients, is a unique candidate for the treatment of diseases associated with ischemias.

However, there is a need for a method and/or a means for the local and/or topical administration of PDGF which allows the angiogenic activity to be increased in vivo in order to obtain a significant density of vessels and which is capable of permitting the formation of a lasting functional neovascular structure.

There is also an unsatisfied need for a method and/or a means for the local and/or topical administration of PDGF which allows the doses of PDGF to be increased in order to stimulate angiogenesis more effectively and overcome the solubility problems previously observed.

The present invention makes it possible to obtain stimulation of angiogenesis as compared with equivalent doses of Regranex. That angiogenic effect is observed during the tissue reconstruction of diabetic rat wounds and is expressed by the hemorrhagic nature of the neoformed tissue, evaluated by a semi-quantitative score established by an independent observer without knowledge of the treatment administered. The angiogenic effect is dose-dependent. In fact, at the same doses as Regranex, intense hemorrhagic phenomena resulting in the premature interruption of administration of the PDGF complex were observed. The observed dose dependence indicates the pharmacological nature of the observed effect.

The observed effect is also confirmed by the histological analysis of the vascular density of the neoformed tissue.

The present invention relates to the use of an amphiphilic polymer in the preparation of a therapeutic composition for promoting angiogenesis at its site of administration, comprising a complex between a polymer and a PDGF, characterized in that the polymer is amphiphilic.

In an embodiment, the present invention relates to the use of an amphiphilic polymer in the preparation of a therapeutic composition for promoting angiogenesis at its site of administration, comprising a complex between an amphiphilic polymer and a PDGF, characterized in that the amphiphilic polymer is selected from the group:
amphiphilic polymers constituted by a hydrophilic polymer skeleton functionalized by hydrophobic substituents and hydrophilic groups, of the general formula I

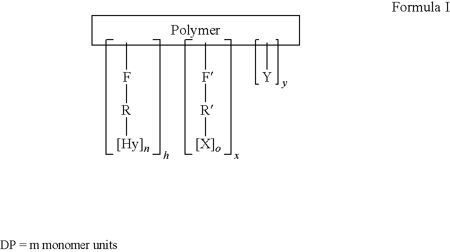

in which
R and R′ are identical or different and represent a bond or a linear, branched and/or unsaturated chain containing from 1 to 18 carbon atoms and optionally containing one or more heteroatoms selected from O, N or/and S,
F and F′ are identical or different and represent a functional group selected from the following functional groups: ester, thioester, amide, carbonate, carbamate, ether, thioether or amine,
X represents a hydrophilic group selected from the group constituted by the following groups:
   carboxylate
   phosphate
   phosphonate,
Y represents a hydrophilic group selected from the group constituted by the following groups:
   phosphate
   phosphonate,
Hy represents a hydrophobic group selected from the group constituted by the following groups:
   linear or branched C₈- to C₃₀-alkyl, optionally unsaturated and/or containing one or more heteroatoms selected from O, N and S,
   linear or branched C₈- to C₁₈-alkylaryl or -arylalkyl, optionally unsaturated and/or containing one or more heteroatoms selected from O, N and S,
   optionally unsaturated C₈- to C₃₀-polycyclic group,
      n and o are from 1 to 3,
      h represents the molar fraction of hydrophobic unit relative to a monomer unit, from 0.01 to 0.5,
      x represents the molar fraction of hydrophilic groups relative to a monomer unit, from 0 to 2.0,
      y represents the molar fraction of hydrophilic groups relative to a monomer unit, from 0 to 0.5.

In an embodiment, the present invention relates to the use of an amphiphilic polymer in the preparation of a therapeutic composition for promoting angiogenesis at its site of administration, comprising a complex between an amphiphilic polymer and a PDGF, characterized in that the amphiphilic polymer is a dextran bifunctionalized by at least one imidazolyl radical Im and at least one hydrophobic group Hy, said radical and group, which are identical and/or different, each being grafted or bonded to the dextran by one or more bonding arms R, Ri or Rh and functional groups F, Fi or Fh,
R being a bond or a chain containing from 1 to 18 carbon atoms, optionally branched and/or unsaturated and containing one or more heteroatoms, such as O, N or/and S,
   R will be called Ri in the case of the imidazoles and Rh in the case of the hydrophobic groups, Ri and Rh being identical or different,
F being an ester, a thioester, an amide, a carbonate, a carbamate, an ether, a thioether, an amine,
   F will be called Fi in the case of the imidazoles and Fh in the case of the hydrophobic groups, Fi and Fh being identical or different,
Im being an imidazolyl radical optionally substituted on one of the carbon atoms by a C₁- to C₄-alkyl group (Alky), of the formula

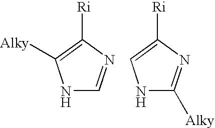

Hy being a hydrophobic group which can be:
   a linear or branched C₈- to C₃₀-alkyl, optionally unsaturated and/or containing one or more heteroatoms, such as O, N or S,
   a linear or branched C₈- to C₃₀-alkylaryl or -aryl-alkyl, optionally unsaturated and/or optionally containing a heteroatom,
   an optionally unsaturated C₈- to C₃₀-polycyclic group.

In an embodiment, the PDGF is selected from the group of the PDGFs (platelet-derived growth factors).

In an embodiment, the complex is characterized in that the PDGF is selected from the group constituted by recombinant human PDGFs having two B chains (rhPDGF-BB).

In an embodiment, the PDGF is PDGF-BB.

The substituents of the amphiphilic polymers constituted by a hydrophilic polymer skeleton functionalized by hydrophobic substituents and hydrophilic groups, of the general formula I

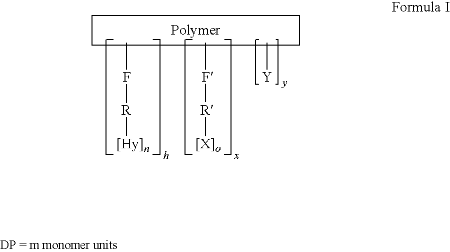

are distributed in a controlled or random manner. Among the polymers having controlled distribution of the substituents there may be mentioned, for example, block copolymers and alternating copolymers.

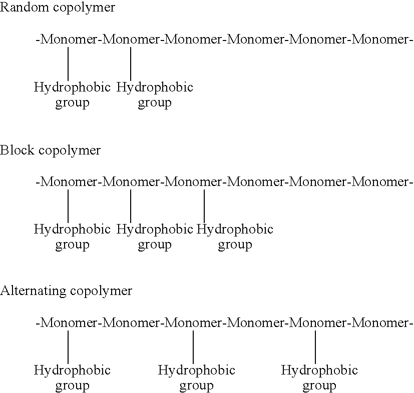

Accordingly, in an embodiment, the polymer is selected from polymers in which the substituents are distributed randomly.

In an embodiment, the amphiphilic polymer is selected from the polyamino acids.

In an embodiment, the polyamino acids are selected from the group constituted by the polyglutamates and the poly-aspartates.

In an embodiment, the polyamino acids are homopoly-glutamates.

In an embodiment, the polyamino acids are homopoly-aspartates.

In an embodiment, the polyamino acids are copolymers of aspartate and glutamate. Those copolymers are either block copolymers or random copolymers.

In an embodiment, the polymer is selected from the poly-saccharides.

In an embodiment, the polysaccharides are selected from the group constituted by hyaluronans, alginates, chitosans, galacturonans, chondroitin sulfate, dextrans, celluloses.

The group of the celluloses is constituted by celluloses functionalized by acids, such as carboxymethylcellulose.

In an embodiment, the polysaccharides are selected from the group constituted by dextrans, hyaluronans, alginates, chitosans.

Those various polysaccharides can be represented as follows:

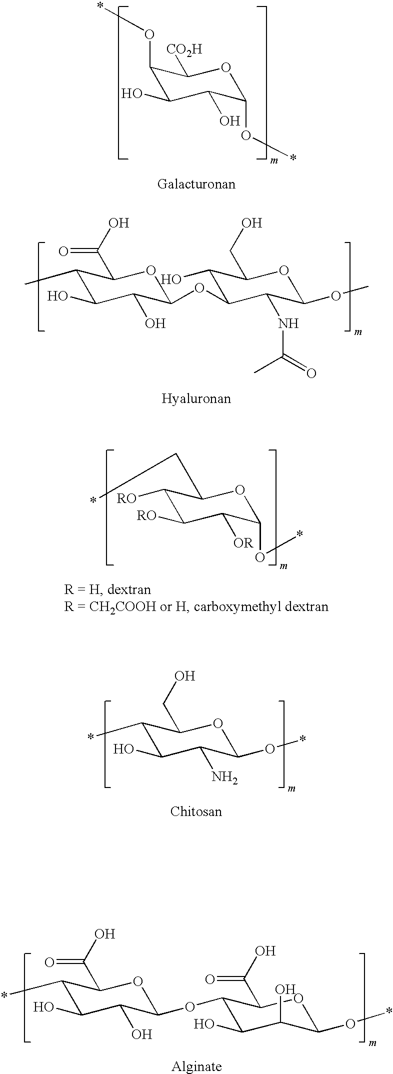

The polysaccharide can have an average degree of polymerization m of from 10 to 10,000.

In an embodiment, it has an average degree of polymerization m of from 10 to 5000.

In another embodiment, it has an average degree of polymerization m of from 10 to 500.

In an embodiment, the hydrophobic group Hy is selected from the group constituted by fatty acids, fatty alcohols, fatty amines, benzylamines, cholesterol derivatives and phenols.

In an embodiment, the cholesterol derivative is cholic acid.

In another embodiment, the phenol is alpha-tocopherol.

The bifunctionalized dextran can correspond to the following general formulae:

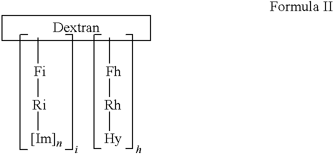

n is from 1 to 3,
i represents the molar fraction of imidazolyl radical relative to a monosaccharide unit, from 0.1 to 0.9,
h represents the molar fraction of hydrophobic group relative to a monosaccharide unit, from 0.01 to 0.5.

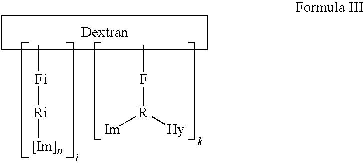

n is from 1 to 3,
i represents the molar fraction of imidazolyl radical relative to a monosaccharide unit, from 0 to 0.9,
k represents the molar fraction of hydrophobic group relative to a monosaccharide unit, from 0.01 to 0.5.

In an embodiment, the dextran in formulae II and III is characterized in that the group Ri, when it is not a bond, is selected from the following groups:

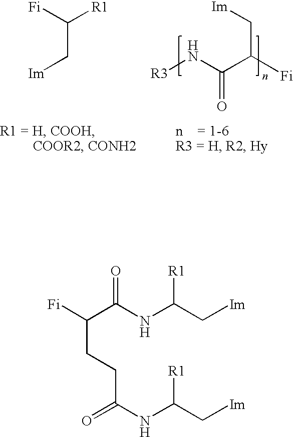

R2 being selected from alkyl radicals containing from 1 to 18 carbon atoms.

In an embodiment, the dextran of formulae II and III is characterized in that the group Ri is a bond.

In an embodiment, the dextran of formulae II and III is characterized in that the group imidazole-Ri is selected from groups obtained by the grafting of a histidine ester, histidinol, histidinamide or histamine.

Those imidazole derivatives can be represented as follows:

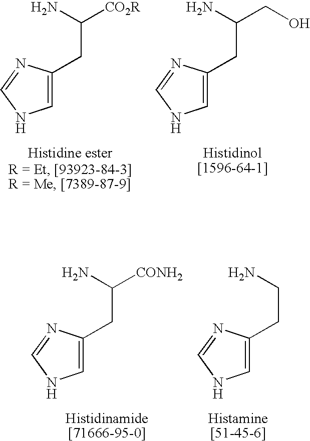

In an embodiment, the dextran of formulae II and III is characterized in that Hy will be selected from the group constituted by fatty acids, fatty alcohols, fatty amines, cholesterol derivatives including cholic acid, phenols including alpha-tocopherol.

In an embodiment, the dextran of formulae II and III is characterized in that the group Rh, when it is not a bond, is selected from the groups:

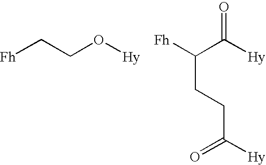

In an embodiment, the dextran of formulae II and III is characterized in that the group Rh is a bond.

In an embodiment, the dextran of formulae II and III is characterized in that the group Ri, when it is not a bond, is selected from the groups

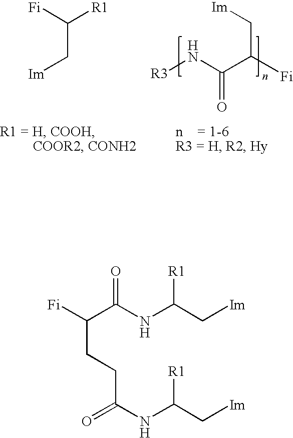

R2 being selected from alkyl radicals containing from 1 to 18 carbon atoms and the group Rh is a bond.

In an embodiment, the dextran of formulae II and III is characterized in that the group imidazole-Ri is selected from histidine esters, histidinol, histidinamide or histamine, and in that Hy will be selected from the group constituted by fatty acids, fatty alcohols, fatty amines, cholesterol derivatives including cholic acid, phenols including alpha-tocopherol.

The dextran of formulae II and III can have a degree of polymerization m of from 10 to 10,000.

In an embodiment, it has a degree of polymerization m of from 10 to 1000.

In another embodiment, it has a degree of polymerization m of from 10 to 500.

The polymers used are synthesized according to techniques known to the person skilled in the art or are purchased from suppliers such as, for example, Sigma-Aldrich, NOF Corp. or CarboMer Inc.

The PDGFs are selected from recombinant human PDGFs obtained according to techniques known to the person skilled in the art or purchased from suppliers such as, for example, Gentaur (USA) or Research Diagnostic Inc. (USA).

The pharmaceutical composition according to the invention is preferably a composition for local and/or topical application which can be in the form of a gel, a cream, a solution, a spray or a paste, or in the form of a patch or a dressing of the active dressing type.

The nature of the excipients which can be formulated with the amphiphilic polymer-PDGF complex according to the invention is chosen in dependence on the form in which it is presented, according to the general knowledge of the galenist.

Accordingly, when the composition according to the invention is in the form of a gel, it is, for example, a gel produced from polymers such as carboxymethylcelluloses (CMCs), vinyl polymers, copolymers of the PEO-PPO type, polysaccharides, PEOs, acrylamides or acrylamide derivatives.

Other excipients can be used in this invention in order to adjust the parameters of the formulation, such as a buffer to adjust the pH, an agent permitting adjustment of the isotonicity, preservatives such as methyl parahydroxy-benzoate, propyl parahydroxybenzoate, m-cresol or phenol, or an antioxidant such as L-lysine hydrochloride.

According to the invention, the therapeutic composition is characterized in that it permits the administration of from 10 μg to 10 mg per ml of PDGF.

In another embodiment, the therapeutic composition permits the administration of from 100 to 1000 μg/ml.

The present invention relates also to the use of an amphiphilic polymer-PDGF complex as defined hereinbefore in the preparation of a therapeutic composition having angiogenic action.

The invention relates also to a therapeutic treatment method for human or veterinary use, characterized in that it comprises the local administration of an angiogenic therapeutic composition comprising a polymer-PDGF complex, characterized in that the polymer is amphiphilic.

In an embodiment, the PDGF is selected from the group of the PDGFs (platelet-derived growth factors).

In another embodiment, the amphiphilic polymer is selected from the group:
amphiphilic polymers constituted by a hydrophilic polymer skeleton functionalized by hydrophobic substituents and hydrophilic groups, of the general formula I

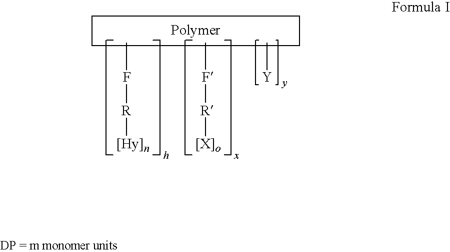

in which
R and R′ are identical or different and represent a bond or a linear, branched and/or unsaturated chain containing from 1 to 18 carbon atoms and optionally containing one or more heteroatoms selected from O, N or/and S,
F and F′ are identical or different and represent a functional group selected from the following functional groups: ester, thioester, amide, carbonate, carbamate, ether, thioether or amine,
X represents a hydrophilic group selected from the group constituted by the following groups:
   carboxylate
   phosphate
   phosphonate,
Y represents a hydrophilic group selected from the group constituted by the following groups:
   phosphate
   phosphonate,
Hy represents a hydrophobic group selected from the group constituted by the following groups:
   linear or branched C₈- to C₃₀-alkyl, optionally unsaturated and/or containing one or more heteroatoms selected from O, N and S,
   linear or branched C₈- to C₁₈-alkylaryl or -arylalkyl, optionally unsaturated and/or containing one or more heteroatoms selected from O, N and S,
   optionally unsaturated C₈- to C₃₀-polycyclic group,
      n and o are from 1 to 3,
      h represents the molar fraction of hydrophobic unit relative to a monomer unit, from 0.01 to 0.5,
      x represents the molar fraction of hydrophilic groups relative to a monomer unit, from 0 to 2.0,
      y represents the molar fraction of hydrophilic groups relative to a monomer unit, from 0 to 0.5,
or from the group of the dextrans bifunctionalized by at least one imidazolyl radical Im and at least one hydro-phobic group Hy, said radical and group, which are identical and/or different, each being grafted or bonded to the dextran by one or more bonding arms R, Ri or Rh and functional groups F, Fi or Fh,
R being a bond or a chain containing from 1 to 18 carbon atoms, optionally branched and/or unsaturated and containing one or more heteroatoms, such as O, N or/and S,
   R will be called Ri in the case of the imidazoles and Rh in the case of the hydrophobic groups, Ri and Rh being identical or different,
F being an ester, a thioester, an amide, a carbonate, a carbamate, an ether, a thioether, an amine,
   F will be called Fi in the case of the imidazoles and Fh in the case of the hydrophobic groups, Fi and Fh being identical or different,
Im being an imidazolyl radical optionally substituted on one of the carbon atoms by a C₁- to C₄-alkyl group (Alky), of the formula

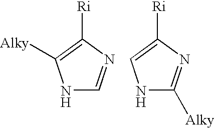

Hy being a hydrophobic group which can be:
   a linear or branched C₈- to C₃₀-alkyl, optionally unsaturated and/or containing one or more heteroatoms, such as O, N or S,
   a linear or branched C₈- to C₃₀-alkylaryl or -aryl-alkyl, optionally unsaturated and/or optionally containing a heteroatom,
   an optionally unsaturated C₈- to C₃₀-polycyclic group.

Examples of the preparation of the various complexes according to the invention are described in patent application IB 2006/002666 in the name of the Applicants.

The use according to the invention makes it possible to obtain results which are said to be dose-dependent in terms of angiogenesis.

The surprising angiogenic activity obtained by the use according to the invention is demonstrated in the examples which follow.

### EXAMPLES

### A Preparation of the Amphiphilic Polymers

### Example 1

### Synthesis of a Succinic Acid Dextran Modified by the Ethyl Ester of Tryptophan

The dextran having an average degree of polymerization of 150, D40, (10 g, Sigma) is dissolved in 25 ml of DMSO at 40° C. To that solution there are added succinic anhydride in solution in DMF (6.2 g in 25 ml) and N-methyl-morpholine, NMM, diluted in DMF (6.2 g in 25 ml). After 1 hour's reaction, the reaction mixture is diluted in water (400 ml) and the polymer is purified by ultrafiltration. The molar fraction of succinic ester formed per glycoside unit is 1.0 according to ¹H-NMR in D₂O/NaOD.

Succinic acid dextran, sodium salt, in aqueous solution (350 g of a solution at 28 mg/ml) is acidified on ion exchange resin (300 ml of moist resin, Purolite, C100H). The resulting solution is frozen and then lyophilized.

Lyophilized succinic acid dextran (8 g) is dissolved in DMF (115 ml) at ambient temperature. The solution is cooled to 0° C., and ethyl chloroformate (3.3 g) and then NMM (3.1 g) are added thereto. The ethyl ester hydrochloride of tryptophan (3.7 g, Bachem) neutralized by TEA (1.4 g) in DMF (37 ml) is then added to the reaction mixture at 4° C., and the mixture is stirred for 45 minutes. After hydrolysis of the remaining activated acids, the polymer is diluted in water (530 ml) and the pH is fixed at 7 by addition of sodium hydroxide solution. The polymer is then purified by ultrafiltration.

The grafting reaction is summarized in the following scheme:

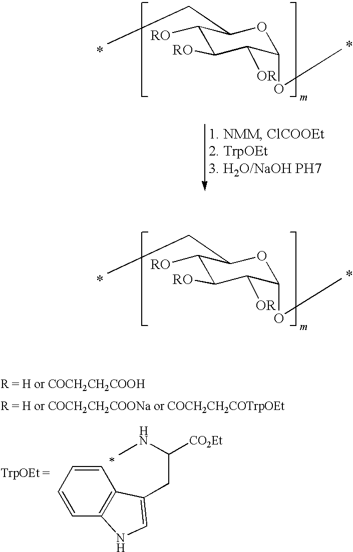

The molar fraction of acids modified by the ethyl ester of tryptophan is 0.45 according to ¹H-NMR in D₂O/NaOD (h=0.45). The molar fraction of unmodified acids per glycoside unit is 0.55 (x=0.55).

### Example 2

### Synthesis of Carboxymethyl Dextran Modified by the Ethyl Ester of Tryptophan

The acids of a carboxymethyl dextran having an average molar mass of about 40 kg/mol (average molar fraction of 1.0 acid per glycoside unit) are activated in the form of mixed anhydrides according to the procedure described in Example 1. The ethyl ester of tryptophan is grafted onto the acids of the polymer according to the procedure described in Example 1. The molar fraction of acids modified by the ethyl ester of tryptophan is 0.45 according to ¹H-NMR in D₂O/NaOD (h=0.45). The molar fraction of unmodified acids per glycoside unit is 0.55 (x=0.55).

### Example 3

### Synthesis of Carboxymethyl Dextran Modified by Tryptophan, Sodium Salt

The polymer obtained in Example 2 is dissolved in water (30 mg/ml) and the pH is fixed at 12.5 by addition of 1N sodium hydroxide solution. After stirring overnight at ambient temperature, the product is purified by ultrafiltration.

The molar fraction of acids modified by the sodium salt of tryptophan is 0.45 according to ¹H-NMR in D₂O (h=0.45).

The molar fraction of unmodified acids per glycoside unit is 0.55 (i=0.55).

### Example 4

### Synthesis of a Carboxymethyl Dextran Modified by Histidinamide and Benzylamine

The acids of a carboxymethyl dextran having an average molar mass of about 40 kg/mol (average molar fraction of 1.0 acid per glycoside unit) are activated in the form of mixed anhydrides according to the procedure described in Example 1. Benzylamine and then histidinamide are added to the solution of activated polymer, and the reaction is carried out at 40° C. for 4 hours.

The grafting reaction is summarized in the following scheme:

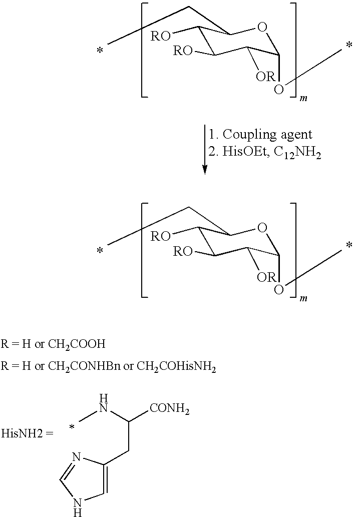

The proportion of acid functional groups modified by:
histidinamide is 55%,
benzylamine is 45%.

The proportion of unmodified acids is zero.

### Example 5

### Synthesis of a Carboxymethyl Dextran Modified by the Ethyl Ester of Histidine and Dodecylamine

The acids of a carboxymethyl dextran having an average molar mass of about 40 kg/mol (average molar fraction of 1.0 acid per glycoside unit) are activated in the form of mixed anhydrides according to the procedure described in Example 1. Dodecylamine and then the ethyl ester of histidine are added to the solution of activated polymer, and the reaction is carried out at 40° C. for 4 hours.

The grafting reaction is summarized in the following scheme:

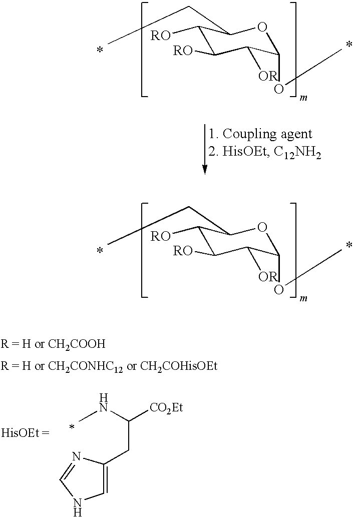

The proportion of acid functional groups modified by:
the ethyl ester of histidine is 85%,
dodecylamine is 10%.

The proportion of unmodified acids is 5%.

### Example 6

### Synthesis of a Carboxymethyl Dextran Modified by Histidinamide and Benzylamine

The acids of a carboxymethyl dextran having an average molar mass of about 40 kg/mol (average molar fraction of 1.0 acid per glycoside unit) are activated in the form of mixed anhydrides according to the procedure described in Example 1. Benzylamine and then histidine are added to the solution of activated polymer, and the reaction is carried out at 40° C. for 4 hours.

The grafting reaction is summarized in the following scheme:

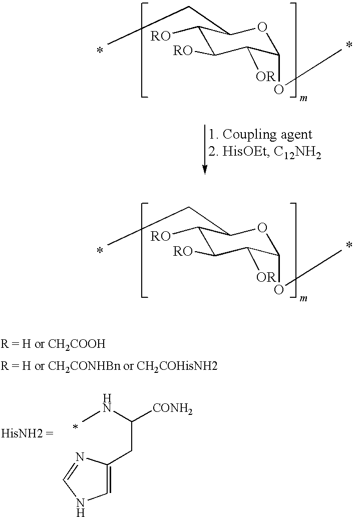

The proportion of acid functional groups modified by:
histidinamide is 65%,
benzylamine is 30%.

The proportion of unmodified acids is 5%.

### Example 7

### Synthesis of a Carboxymethyl Dextran Modified by the Ethyl Ester of Histidine and Benzylamine

The acids of a carboxymethyl dextran having an average molar mass of about 40 kg/mol (average molar fraction of 1.0 acid per glycoside unit) are activated in the form of mixed anhydrides according to the procedure described in Example 1. Benzylamine and then the ethyl ester of histidine are added to the solution of activated polymer, and the reaction is carried out at 40° C. for 4 hours.

The grafting reaction is summarized in the following scheme:

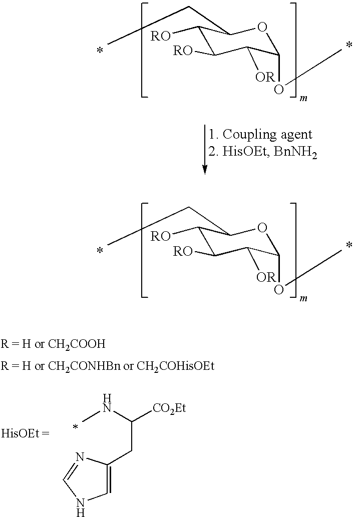

The proportion of acid functional groups modified by:
the ethyl ester of histidine is 10%,
benzylamine is 45%.

The proportion of unmodified acids is 45%.

### Example 8

### Synthesis of a Carboxymethyl Dextran Modified by the Ethyl Ester of Histidine and Benzylamine

The acids of a carboxymethyl dextran having an average molar mass of about 40 kg/mol (average molar fraction of 1.0 acid per glycoside unit) are activated in the form of mixed anhydrides according to the procedure described in Example 1. Benzylamine and then the ethyl ester of histidine are added to the solution of activated polymer, and the reaction is carried out at 40° C. for 4 hours.

The grafting reaction is summarized in the following scheme:

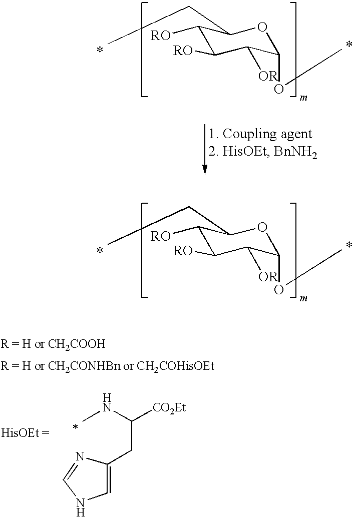

The proportion of acid functional groups modified by:
the ethyl ester of histidine is 20%,
benzylamine is 45%.

The proportion of unmodified acids is 35%.

### Example 9

### Synthesis of an Alginate Modified by Dodecylamine

Product prepared according to patent FR2781677, having the following formula:

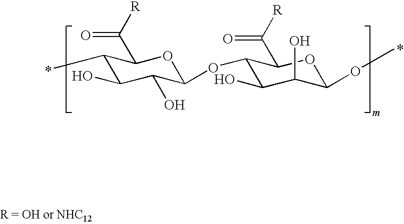

The proportion of acid functional groups modified by: dodecylamine is 10%.

### B Preparation of the Complexes

The preparation of the complexes is carried out under a laminar flow hood in an area with a controlled atmosphere. The PDGF-BB is produced by Peprotech or hnExport. The PDGF-BB is produced either in yeast (Saccharomyces cerevisiae) or in bacteria (Escherichia coli).

### Example 10

### Preparation of a PDGF-BB/Polymer Complex 1/100 mg/ml

In a sterile Falcon tube, 7.1 mg of lyophilized PDGF-BB are dissolved in 3.55 ml of 10 mM sodium acetate buffer, pH 5. In a second tube, 3.14 g of the amphiphilic polymer obtained in Example 2 are dissolved in 11.5 g of sterile water to which there are added a solution of sterile water containing 0.9% NaCl, a solution of bidistilled sterile water and a 1N NaOH solution in order to adjust the polymer concentration to 200 mg/ml, the pH to 7.4 and the osmolality to 300 mOsm. After homogenization of the two solutions, the 3.55 ml of the first solution are added to 3.55 ml of the second solution in order to obtain a complex in which the concentration of PDGF-BB is 1 mg/ml and that of the polymer is 100 mg/ml. The resulting solution is filtered over 0.22 μm before being distributed into two sterile Falcon tubes.

### Example 11

### Preparation of a PDGF-BB/Polymer Complex 1/50 mg/ml

In a sterile Falcon tube, 7.1 mg of lyophilized PDGF-BB are dissolved in 3.55 ml of 10 mM sodium acetate buffer, pH 5. In a second tube, 1.57 g of the amphiphilic polymer obtained in Example 2 are dissolved in 11.5 g of sterile water to which there are added a solution of sterile water containing 0.9% NaCl, a solution of bidistilled sterile water and a 1N NaOH solution in order to adjust the polymer concentration to 100 mg/ml, the pH to 7.4 and the osmolality to 300 mOsm. After homogenization of the two solutions, the 3.55 ml of the first solution are added to 3.55 ml of the second solution in order to obtain a complex in which the concentration of PDGF-BB is 1 mg/ml and that of the polymer is 50 mg/ml. The resulting solution is filtered over 0.22 μm before being distributed into two sterile Falcon tubes.

### Example 12

### Preparation of a PDGF-BB/Polymer Complex 2/100 mg/ml

In a sterile Falcon tube, 14.2 mg of lyophilized PDGF-BB are dissolved in 3.55 ml of 10 mM sodium acetate buffer, pH 5. In a second tube, 3.14 g of the amphiphilic polymer obtained in Example 2 are dissolved in 11.5 g of sterile water to which there are added a solution of sterile water containing 0.9% NaCl, a solution of bidistilled sterile water and a 1N NaOH solution in order to adjust the polymer concentration to 200 mg/ml, the pH to 7.4 and the osmolality to 300 mOsm. After homogenization of the two solutions, the 3.55 ml of the first solution are added to 3.55 ml of the second solution in order to obtain a complex in which the concentration of PDGF-BB is 2 mg/ml and that of the polymer is 100 mg/ml. The resulting solution is filtered over 0.22 μm before being distributed into two sterile Falcon tubes.

### Example 13

### Preparation of a PDGF-BB/Polymer Complex 4/100 mg/ml

In a sterile Falcon tube, 28.4 mg of lyophilized PDGF-BB are dissolved in 3.55 ml of 10 mM sodium acetate buffer, pH 5. In a second tube, 3.14 g of the amphiphilic polymer obtained in Example 2 are dissolved in 11.5 g of sterile water to which there are added a solution of sterile water containing 0.9% NaCl, a solution of bidistilled sterile water and a 1N NaOH solution in order to adjust the polymer concentration to 200 mg/ml, the pH to 7.4 and the osmolality to 300 mOsm. After homogenization of the two solutions, the 3.55 ml of the first solution are added to 3.55 ml of the second solution in order to obtain a complex in which the concentration of PDGF-BB is 4 mg/ml and that of the polymer is 100 mg/ml. The resulting solution is filtered over 0.22 g/m before being distributed into two sterile Falcon tubes.

### C Demonstration of the Angiogenic Activity of the Complexes

The surprising angiogenic activity obtained by the use of the PDGF complex according to the invention is demonstrated in an in vivo model of cutaneous scarring.

The in vivo tests were carried out on diabetic db/db rat wounds. The groups comprise a minimum of 4 wounds. On the first day, two excisions of 2.5×2.5 cm² were made on the rat's back.

### Example 14

### Increase in the Angiogenic Response with the PDGF-BB Complex Relative to the Commercial PDGF-BB Formulation

According to the protocol, the formulations were to be applied to the wounds every 2 days for 22 days, after cleaning the wound. The reference group is treated with the commercial PDGF-BB formulation in gel form, Regranex® (Johnson & Johnson), at a dose of 500 μl per application. The group treated with the complex described in Example 10 received a dose of 100 μl, that is to say twice as much PDGF-BB as in the group treated with Regranex.

The hemorrhagic score is evaluated by visual observation on a qualitative linear scale of from 0 to 4, 0 representing the absence of bleeding and 4 representing maximum bleeding. On the 8th day after the excision and the start of treatment, that is to say after 4 applications of the products, the score reaches on average 2.8 for the group treated with the PDGF-BB complex, as compared with 1.3 for the group treated with Regranex. This difference is significant from a statistical point of view.

The hemorrhagic score of 2.8 on average in the group treated with the PDGF-BB complex required the treatment to be discontinued after 4 applications, while treatment with Regranex could be continued up to the 22nd day as intended.

This example shows that it is advantageous to increase the doses of PDGF-BB in order to increase the angiogenesis, which was not reported with the product Regranex.

### Example 15

### Angiogenic Effect of the Complex Dependent on the Applied Dose of PDGF-BB

A PDGF-BB complex formulation as described in Example 10 was applied to the wounds according to 2 different protocols. Group 1 comprises 2 applications of 100 μl on day 0 and 90 μl on day 2, after cleaning the wound. The wounds were then simply cleaned with a saline solution every 2 days for 16 days. Group 2 comprises 4 applications of 100 μl on day 0, 90 μl on day 2, 80 μl on day 4 and 70 μl on day 6. The wounds were then cleaned with a saline solution every 2 days for 16 days. The volume of PDGF-BB complex solution decreases in order to maintain a constant dose per unit surface area, taking into account the reduction in the surface area of the wound.

On the 10th day after the excision and the start of treatment, the hemorrhagic score reaches on average 2.2 for the group treated 4 times with the PDGF-BB complex, as compared with 1.4 for the group treated 2 times with the same PDGF-BB complex. This difference is significant from a statistical point of view. The PDGF-BB complex allows an angiogenic activity dependent on the applied dose to be displayed, in contrast with Regranex, for which no dose-related effect is reported in the literature.

### Example 16

### Angiogenic Effect of the Complex Dependent on the Applied Dose of PDGF-BB

Three formulations of complexes with concentrations of PDGF-BB of 1, 2 and 4 mg/ml were prepared with the same amphiphilic polymer at a concentration of 100 mg/ml as described in Examples 10, 12 and 13. After cleaning the wounds, treatment with the three PDGF-BB complex formulations is the same and comprises 3 applications of 90 μl on day 0, 65 μl on day 2 and 55 μl on day 4.

The hemorrhagic score measured on day 7 shows an effect dependent on the dose of PDGF-BB with the complex. In fact, a single dose gives a hemorrhagic score of 1, double the dose gives 2.1 and four times the dose gives 2.5.

### Example 17

### Angiogenic Effect of the PDGF-BB Complex Formulation Compared with the Commercial PDGF-BB Formulation

Treatment with the PDGF-BB complex described in Example 11 comprises the application of 100 μl on day 0 and 90 μl on day 2, after cleaning the wound. The wounds were simply cleaned with a saline solution on day 4. Treatment with Regranex comprises 3 applications of 500 μl on day 0, 450 μl on day 2 and 400 μl on day 4, after cleaning the wound. The rats were sacrificed on day 6 and histological sections of the wounds were prepared.

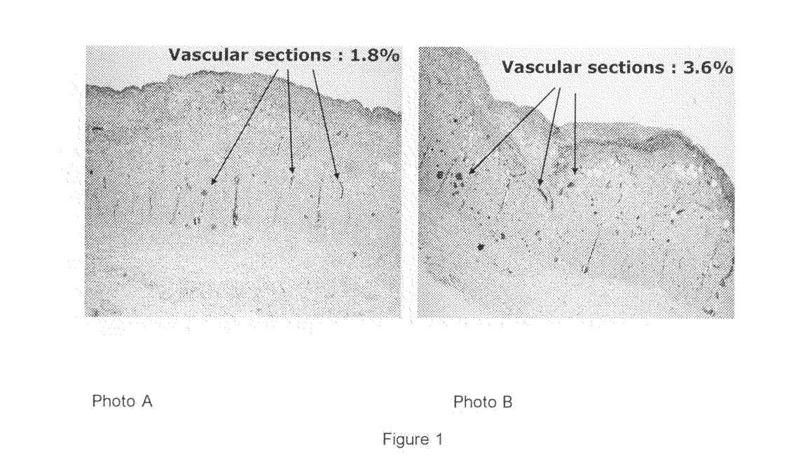

The photographs of the histological sections are shown in FIG. 1. Quantification by image analysis allows the surface area of the new vessels formed, relative to the surface area of neoformed dermis, to be estimated at 3.6% in the case of the PDGF-BB complex (Photo B) and at 1.8% for Regranex (Photo A). This histological analysis on day 6 shows the superior angiogenic ability of the PDGF-BB complex as compared with a simple formulation of the protein.

1. A therapeutic method for promoting angiogenesis in a human or animal at a local site of administration of an angiogenic therapeutic composition, the method comprising:
administrating the angiogenic composition locally to the site of the human or animal, the angiogenic composition comprising a complex between an amphiphilic polymer and a platelet-derived growth factor (PDGF), wherein angiogenesis is promoted at the site of administration of the angiogenic composition.

2. The method of claim 1, wherein the PDGF is selected from the group of the PDGFs (platelet-derived growth factors).

3. The method of claim 1, wherein the amphiphilic polymer is selected from the group consisting of:
(a) amphiphilic polymers comprising a hydrophilic polymer skeleton functionalized by hydrophobic substituents and hydrophilic groups, of formula I: 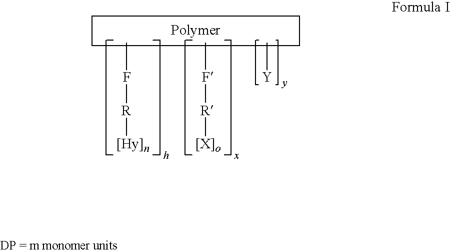
where:
R and R′ are identical or different and represent a bond or a linear, branched, and/or unsaturated chain containing from 1 to 18 carbon atoms and optionally contains one or more heteroatoms selected from O, N, and S;
F and F′ are identical or different and each represents a functional group selected from the group consisting of esters, thioesters, amides, carbonates, carbamates, ethers, thioethers, and amines;
X represents a hydrophilic group selected from the group consisting of carboxylates, phosphates, and phosphonates;
Y represents a hydrophilic group selected from the group consisting of phosphates and phosphonates;
Hy represents a hydrophobic group selected from the group consisting of:
linear or branched C₈- to C₃₀-alkyls, optionally unsaturated and/or containing one or more heteroatoms selected from O, N and S,
linear or branched C₈- to C₁₈-alkylaryls or -arylalkyls, optionally unsaturated and/or containing one or more heteroatoms selected from O, N and S, and
C₈- to C₃₀-polycyclic groups, optionally unsaturated;
n and o are from 1 to 3;
h represents a molar fraction of hydrophobic units relative to a monomer unit in a range from 0.01 to 0.5;
x represents a molar fraction of hydrophilic groups relative to a monomer unit, in a range from 0 to 2.0; and
y represents the molar fraction of hydrophilic groups relative to a monomer unit, from 0 to 0.5; and
(b) dextrans bifunctionalized by at least one imidazolyl radical Im and at least one hydrophobic group Hy, wherein the radical and group are identical or different, and each is grafted or bonded to the dextran by one or more bonding arms R, Ri, or Rh, and functional groups F, Fi, or Fh,
where:
R is a bond or a chain containing from 1 to 18 carbon atoms, optionally branched and/or unsaturated and optionally containing one or more heteroatoms selected from O, N, and S, wherein R is called Ri in the case of the imidazoles and Rh in the case of the hydrophobic groups, Ri and Rh being identical or different;
F is an ester, a thioester, an amide, a carbonate, a carbamate, an ether, a thioether, or an amine, wherein F is called Fi in the case of the imidazoles and Fh in the case of the hydrophobic groups, Fi and Fh being identical or different;
Im is an imidazolyl radical optionally substituted on one of the carbon atoms by a 01- to 04-alkyl group (Alky), of the formula 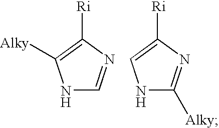
and
Hy is a hydrophobic group selected from the group consisting of:
linear or branched C8- to CH-alkyls, optionally unsaturated and/or containing one or more heteroatoms selected from O, N, and S,
linear or branched C8- to C30-alkylaryls or -aryl-alkyls, optionally unsaturated and optionally containing a heteroatom, and
saturated and unsaturated C8- to C30-polycyclic groups.

4. The method of claim 1, wherein the PDGF is selected from the group consisting of recombinant human PDGFs having two B chains (rhPDGF-BB).

5. The method of claim 1, wherein the PDGF is PDGF-BB.

6. The method of claim 1, wherein the composition is administered in an amount of from 10 μg to 10 mg per ml of PDGF.

7. The method of claim 1, wherein the therapeutic composition is in the form of a gel, a cream, a solution, a spray, a paste, a patch, or a dressing.
